(19) United States
(12) Patent Application Publication
(10) Pub. No.: US 2024/0358239 A1
(43) Pub. Date: Oct. 31, 2024

(54) Endoscopic Bite Block With Compressible Bite Surface

(71) Applicant: Anish Sheth, Princeton, NJ (US)

(72) Inventors: Anish Sheth, Princeton, NJ (US); William Douglas Hare, Princeton, NJ (US)

(73) Assignee: Anish Sheth, Princeton, NJ (US)

(21) Appl. No.: 18/649,911

(22) Filed: Apr. 29, 2024

Related U.S. Application Data
(60) Provisional application No. 63/498,641, filed on Apr. 27, 2023.

(51) Int. Cl. A61B 1/00; A61B 1/273; A61M 25/06

(52) U.S. Cl. CPC A61B 1/00154; A61B 1/2736; A61M 2025/0681

(57) ABSTRACT

The bite block for endoscopic procedures includes a tubular body, a front plate, a movable surface member and an elastic member. The tubular body has an internal lumen configured to receive an endoscope, and has an upper outer surface and a lower outer surface. The front plate has an upper wall and a lower wall, both connected to the tubular body and the upper wall extending in a first direction and the lower wall extending in a second generally opposite direction. The movable surface member is mounted to one of the upper outer surface and the lower outer surface of the tubular body and is configured to move relative to the tubular body. The elastic member is configured to join the movable surface member and the tubular body. The elastic member exerts a force against movement of the movable surface member in the direction of the tubular body.

### TECHNICAL FIELD OF THE INVENTION

The technical field of the invention relates to endoscopic bite blocks that include a bite surface that is movable relative to a base surface.

### BACKGROUND OF THE INVENTION

During endoscopic procedures an endoscope is passed through a patient's mouth, through the throat and into the stomach. To facilitate use of the endoscope, an endoscopic bite block is placed in the patient's mouth between the upper and lower teeth. A strap that passes around the back of the patient's head is used to hold the bite block in place within the patient's mouth. The bite block has an opening through which the physician passes the endoscope. Each side of the opening has a wing to which an opposite end of the strap is attached.

FIGS. 1 and 2 are front and rear perspective views, respectively, of a conventional bite block 10. Conventional bite blocks 10 are generally characterized by a number of structural features. First, the bite block includes the tubular body or barrel 12 whose internal lumen or channel 18 serves as the channel for passage of the endoscope, and whose top and bottom outer surfaces 30, 32 serve as the surfaces upon which the subject's teeth bite. These surfaces are generally flattened. The tubular body 12 has a proximal end 14 and a distal end 16, and upper and lower surfaces 30, 32. A channel 18 extends through the tubular body 12. The endoscope (not shown) or other instruments may be inserted through this channel into the patient's throat.

Second, the bite block 10 includes an upper wall 20 and a lower wall 21, both centrally connected to the outer end of the tubular body 12, as well as left and right lateral wings 22, 24, respectively, shaped such that the wall and wings should rest comfortably adjacent to and around the subject's mouth and cheeks. It is these walls that provide a general alignment direction to the tubular body 12 and prevent the bite block from slipping into the mouth. This combination of the walls 20, 21 and wings 22, 24 is also known as the front plate.

Third, each wing 22, 24 has a tab or clip 26, 28. An elastic band (not shown) may be attached to the clips 26, 28 and passed around the patient's head to secure the bite block firmly to the subject's head. The tubular body 12 may further include upper and lower ridges 38 that are present as an obstruction to the bite block sliding out of the patient's mount.

Because of patient discomfort in passing an endoscope through a patient's mouth and throat, the patient is anesthetized to some degree. The bite block can become malpositioned during the process of anesthetizing the patient when it is common for the patient to have an involuntary jaw opening (e.g., yawning) motion. This jaw opening motion can cause the bite block to slip out of the patient's mouth or become disoriented within the teeth and mouth.

The bite block can also become malpositioned during times when anesthesia is inadequate and the patient experiences agitation. During these times, the patient will often try to open their mouth and push out the bite block with their tongue, or they will open their mouth in an attempt to speak to the physician.

If the bite block becomes disoriented during jaw opening, mouth trauma can occur when the jaw is closed on a malpositioned bite block. Bite block malposition has been associated with injury to the gums and palate, and most significantly, dental injury.

### SUMMARY OF THE INVENTION

In one general aspect the invention is a bite block for use during endoscopic procedures. The bite block includes a tubular member, a front plate, at least one movable surface member, and at least one flexible member.

The tubular body has an internal lumen configured to receive an endoscope, and has an upper outer surface and a lower outer surface. The front plate includes an upper wall and a lower wall, both connected to an outer end of the tubular body and the upper wall extending in a first direction and the lower wall extending in a second generally opposite direction. The movable surface member is mounted to one of the upper outer surface and the lower outer surface of the tubular body and is configured to move relative to the tubular body. At least one flexible member is mounted at a first end to the movable surface member and at a second end to the tubular body. The flexible member is configured to exert a force against movement of the movable surface member in the direction of the tubular body.

Embodiments of the bite block may include one or more of the following features. For example, the bite block may further include a strap or band connected to the bite block and configured to retain the bite block to a subject's head.

The bite block may further include at least one of an upper ridge and a lower ridge on the upper movable surface member and the lower movable surface member, respectively, and configured to interact with the subject's teeth to resist the bite block from sliding out of a subject's mouth in use.

The bite block may include an upper movable surface member and a lower movable surface member.

The movable surface member comprises a first portion having a first end and a second opposite end and is generally flat. The movable surface member may further include a first side portion extending from the first end and generally perpendicular to the first portion and a second side portion extending from the second end and generally perpendicular to the first portion and parallel to the first side portion.

The flexible member may include one or more of an elastic spring, an elastic hinge and an elastic strip. The flexible member may be one or more elastic hinges.

The front plate may include one or more of a groove, flange or tab and the movable surface may include one or more of a groove, flange or tab configured to mate with the groove, flange or tab on the front plate. Mating of the groove, flange or tab between the front plate and movable surface may guide the movement of the movable surface relative to the front plate or may retain the movable surface to the front plate during movement of the movable surface relative to the front plate.

An outer surface of the tubular body may include one or more of a groove, flange or tab and the movable surface may include one or more of a groove, flange or tab configured to mate with the groove, flange or tab on the tubular body. The mating of the groove, flange or tab between the tubular body and movable surface may guide the movement of the movable surface relative to the tubular body or may retain the movable surface to the tubular body during movement of the movable surface relative to the tubular body.

### DESCRIPTION OF THE DRAWINGS

FIG. 1 is a front perspective view of a conventional endoscopic bite block.

FIG. 2 is a rear perspective view of a conventional endoscopic bite block.

FIGS. 3 and 4 are front and rear perspective views, respectively, of the first embodiment of a modified endoscopic bite block.

FIGS. 5 and 6 are front and rear views, respectively, of the flexible member of the modified endoscopic bite block of FIGS. 3 and 4.

FIGS. 7 and 8 are front and rear perspective views, respectively, of the second embodiment of a modified endoscopic bite block.

FIG. 9 is a front view of the flexible member of the second embodiment of a modified endoscopic bite block.

FIG. 10 is a schematic diagram of the hinges of the flexible member of the second embodiment of a modified endoscopic bite block.

FIGS. 11 and 12 are front and rear perspective views, respectively, of the third embodiment of a modified endoscopic bite block.

FIG. 13 is a front view of the flexible member of a modification of the second embodiment of a modified endoscopic bite block.

FIG. 14 is an end view of the bite portion of the bite block showing a fourth embodiment of an upper compressible bite surface.

FIG. 15 is an end view of the bite portion of the bite block showing a fifth embodiment of an upper compressible bite surface.

FIG. 16 is an end view of the bite portion of the bite block showing a sixth embodiment of a bite block having upper and lower compressible bite surfaces.

FIG. 17 is a perspective view of an elastic hinge that may be used in the modified bite blocks.

FIG. 18 is a perspective view of an elastic strip that may be used in the modified bite blocks.

FIG. 19 is a perspective view of an elastic spring that may be used in the modified bite blocks.

FIGS. 20 and 21 are side views of a bowed elastic hinge and a bowed elastic strip that may be used in the modified bite block.

FIG. 22 is a schematic end view of an endoscopic bite block with grooves, flanges or tabs for retaining or guiding the movable surface member to the bite block.

FIGS. 23 and 24 are top views of mating embodiments of the T-shaped grooves and flanges for use in the endoscopic bite block of FIG. 22.

FIGS. 25 and 26 are top views of mating embodiments of the tab-shaped movable surface member illustrating a tab-shaped groove and a tab-shaped protrusion.

FIGS. 27 and 28 are end views of the movable surface member illustrating mating grooves, flanges or tabs to mate with the groves, flanges or tabs of FIGS. 23-26.

FIG. 29 is an end view of the second embodiment of an endoscopic bite block modified to include elastic members in the form of springs.

### DESCRIPTION OF THE INVENTION

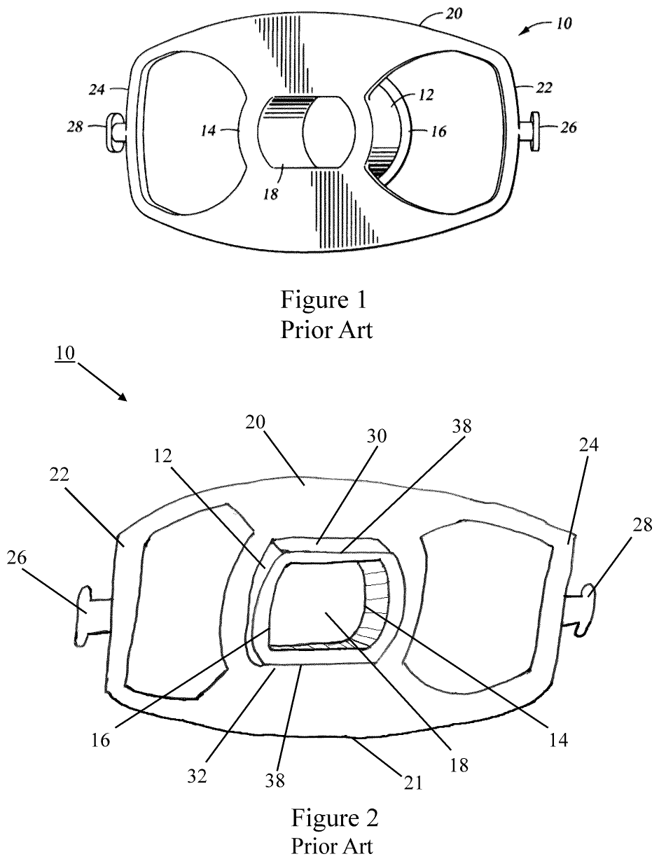

As discussed in detail below, to address the jaw opening motion discussed above, variations of the bite block 10 are provided with one or more movable surfaces that either the upper teeth, lower teeth, or both, rest upon. FIGS. 1 and 2 illustrate surfaces that a patient may rest his teeth on. Those surface are stationary relative to the endoscopic bite block. In contrast, the modified bite blocks include one or more surfaces that are configured to follow the movement of a patient's teeth resting upon the surfaces. In this manner, when a patient yawns, the surface will move in the same direction as the patient's teeth to prevent dislodging the bite block from the patient's mouth. It should be understood that the modified endoscopic bite block can include an upper movable surface and a stationary surface, a lower movable surface and a stationary upper surface, or an upper movable surface and a lower movable surface.

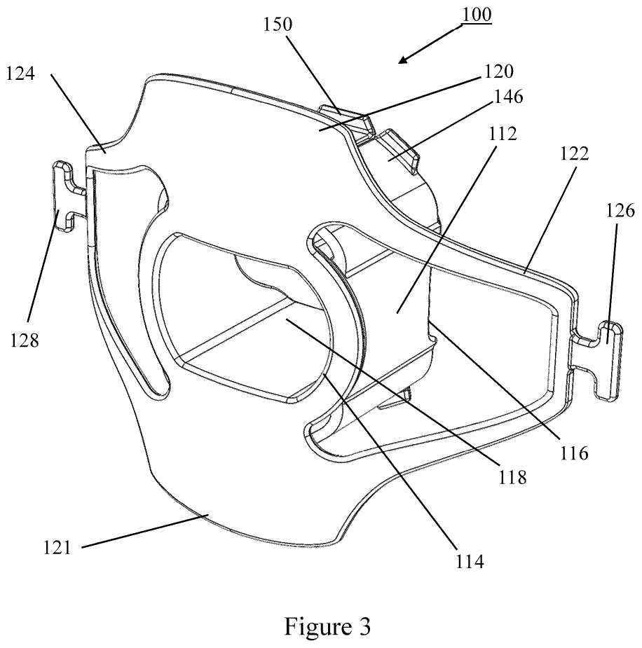

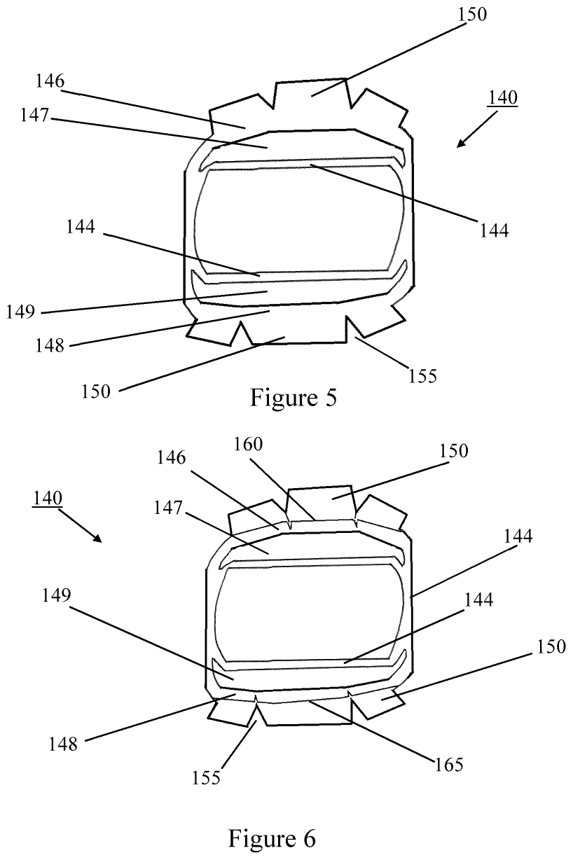

Referring to FIGS. 3-6, a first embodiment of a modified bite block 100 includes a tubular body or barrel 112 that defines a channel 118 that passes between proximal and distal openings 114, 116, respectively. The barrel 112 includes upper surface 130 and lower surface 132. The bite block 100 also includes upper and lower walls 120, 121, respectively, lateral wings 122, 124, and clips 126, 128. A flexible, expandable member 140 is positioned around the outer surfaces of the barrel 112 (i.e., around upper and lower surfaces 130, 132 and side surfaces of the barrel).

The member 140 includes a tubular portion 144 that surrounds the outer surface of the barrel 112 and includes an upper portion 146 and a lower portion 148. The upper and lower portions 146, 148 are biased away from the tubular portion 144 and each creates a respective channel 147, 149. The channel 147 is defined by the tubular portion 144 and the upper portion 146 and the channel 149 is defined by the tubular portion 144 and the lower portion 148. Because of the bias within the upper and lower portions 146, 148, when they are compressed in the direction of the tubular portion 144 by application of force, the volume of channels 147, 149 will be reduced. Upon release of the compressive force, the upper and lower portions 146, 148, their bias will cause them to expand outwardly, away from the tubular portion 144.

The upper and lower portions each include one or more ridge members 150. FIGS. 3-6 disclose three ridge member 150 on each of the upper and lower portions. However, fewer or more ridge members may be used. Each ridges member is separated from an adjacent ridge member by a gap 155. When the upper and lower portions are compressed inwardly in the direction of the tubular portion 144, the area of each gap 155 will be reduced because the ridge members would tend to form a continuous ridge upon compression.

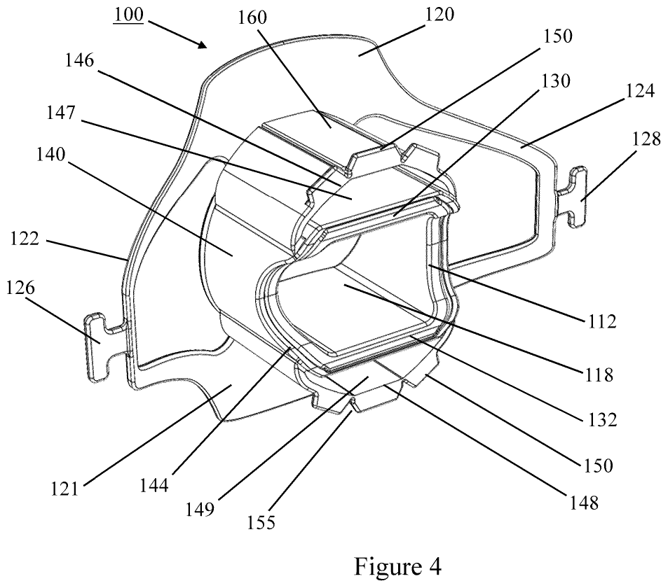

FIGS. 3 and 4 show that the gaps 155 between the ridge members 150 extends from the ridge members to the opposite end of the upper and lower portions 146, 148. In this manner, the area of gaps 155 formed in the upper and lower portions is reduced upon compression because the upper and lower portions would tend to form continuous surfaces upon application of a compressive force.

In use during an endoscopic procedure, the endoscopic bite block 100 will be positioned such that the tubular member 112 is inserted into a patient's mouth with the patient's upper and lower teeth resting upon upper and lower surfaces 160, 165, of the upper and lower portions 146, 148, respectively, between the front plate and the ridge members 150. The flexible member 140 is configured such that during use (i.e., when a patient's teeth are resting on the upper and lower surfaces 160, 165) there is no distance between the upper portion and the tubular portion and the lower portion and the tubular portion, or that distance is at least minimized. In this manner, when a patient's jaw opening motion occurs, as previously described, the bias of the member 140 will cause the upper and lower portions to follow the movement of the upper and lower teeth, respectively. This movement tends to keep upper and lower portions in contact with the patient's teeth, which prevents the bite block from dislodging from the patient's mouth.

The flexible member 140 may be injection molded using an elastic polymer such as a urethane, nylon, urethane/nylon blend, or other single polymer or mixture of polymers. In addition to being elastic, the polymer should be biocompatible, acceptable for medical use, and suitable for use in a patient's mouth. One suitable polymer may be a Pebax® polymer, which is a block copolymer of polyamide and polyether blocks. In one implementation, the flexible member 140 may be made as a separate part that then is placed over the tubular body or barrel 112. Because of the elasticity of the flexible member 140, the tubular portion 144 can be stretched around the tubular body 112 and allowed to be compressed around the tubular body 112. In this implementation, individual flexible members may be configured with variations and selected for individual patients. In another implementation, the flexible member 140 may be injection molded around the tubular body 112 and thereby be integral with the bite block 100.

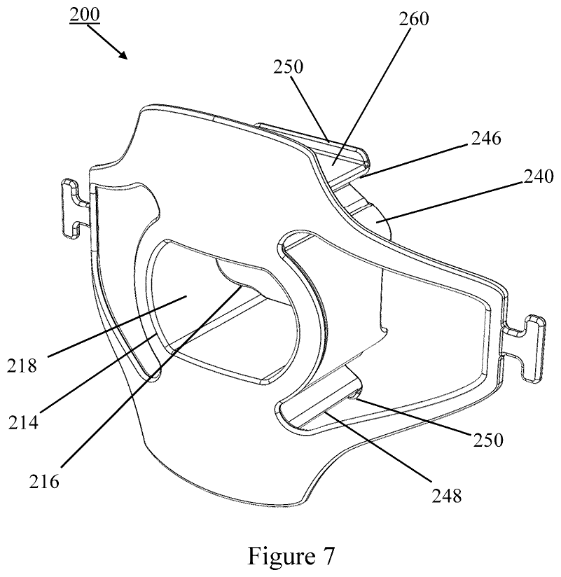

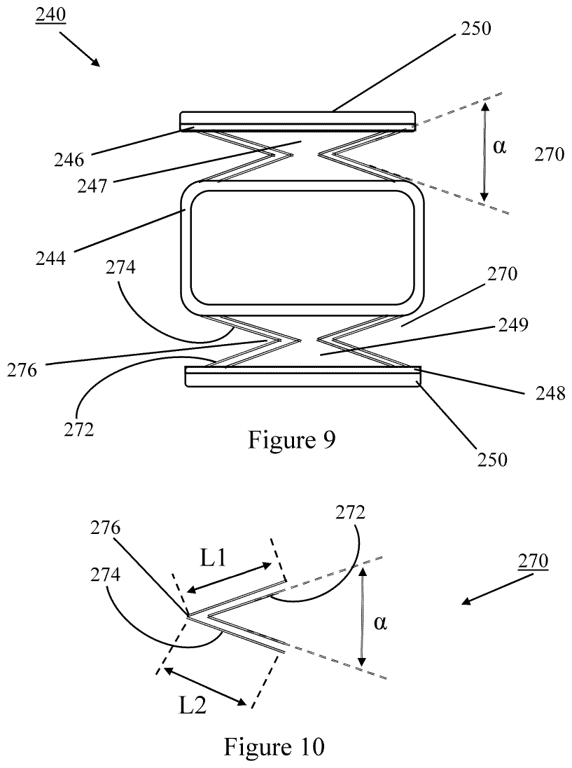

Referring to FIGS. 7-9, a second embodiment of a modified bite block 200 includes a tubular body or barrel 212 that defines a channel 218 that passes between proximal and distal openings 214, 216, respectively. The bite block 200 includes many of the features of the bite block 100, such as upper and lower surfaces of the barrel 212. upper and lower walls, lateral wings, and clips. A flexible, expandable member 240 is positioned around the outer surfaces of the barrel 212 (i.e., around the side, upper and lower surfaces). The difference between bite block 100 and bite block 200 is between the flexible member 140 and a flexible member 240 of bite block 200.

The flexible member 240 includes a tubular portion 244 that surrounds the outer surfaces (upper, lower and side surfaces) of the barrel 212 and includes an upper portion 246 and a lower portion 248. The upper and lower portions 246, 248 are biased away from the tubular portion 244 and each creates a respective channel 247, 249. The channel 247 is defined by the tubular portion 244 and the upper portion 246 and the channel 249 is defined by the tubular portion 244 and the lower portion 248. Because of the bias within the upper and lower portions 246, 248, when they are compressed in the direction of the tubular portion 244 by application of force, the volume of channels 247, 249 will be minimized. Upon release of the compressive force, the upper and lower portions 246, 248 will expand away from the tubular portion 244.

In contrast to the bite block 100, the bite block 200 includes a single ridge member 250 on each of the upper and lower portions. Also in contrast to bite block 100, bite block 200 includes a pair of flexible hinges 270 between each of the upper and lower portions 246, 248 and the tubular portion 244 on each side of the bite block. The hinges 270 are defined by a pair of walls 272, 274 that join together at one end to form an angled joint 276. At opposite ends of the walls from the angled joint, the walls are connected to the tubular portion 244 or the upper and lower portions 246, 248. Specifically, for the hinge 270 on the upper half of the bite block 200, the wall 272 is connected at one end to the tubular portion 244 and at the opposite end to the wall 274 at angled joint 276. The wall 274 is connected at one end to the upper portion 246 and at the opposite end to the wall 272 at the angled joint 276.

Each pair of wall members 272, 274 are separated at the end opposite of the angled joint by a gap 255. When the upper and lower portions 246, 248 are compressed in the direction of the tubular portion 244, the width of each gap 255 is reduced as the angle of the angled joint is reduced.

In use during an endoscopic procedure, the endoscopic bite block 200 will be positioned such that the tubular member 212 is inserted into a patient's mouth with the patient's upper and lower teeth resting upon the upper and lower portions 246, 248, respectively, between the front plate and the ridge members 250. The member 240 is configured such that during use there is no distance between the upper portion and the tubular portion and the lower portion and the tubular portion, or that distance is at least minimized. In this manner, when a patient's jaw opening motion occurs, as described above, the bias of the member 240 will cause the upper and lower portions to follow the movement of the upper and lower teeth, respectively. This following movement tends to keep upper and lower portions in contact with the patient's teeth, which prevents the bite block from dislodging from the patient's mouth.

The modified bite block 200 can be fabricated in the same manner as described above for the bite block 100. The flexible member 240 can be a separate article from the bite block or an integral portion of the bite block 200. If it is a separate article, the tubular portion 244 can be placed around the tubular body 212 and its elasticity will hold the flexible member in place on the tubular body.

FIG. 10 provides a schematic diagram of the hinge 270. The hinge 270 can be modified by varying the length L1 of the wall 272 and the length L2 of the wall 274 independently of each other such that L1≠L2 or L1=L2. Further, the angle alpha (α) of the joint 276 can be varied as desired over a range of about 0 degrees to about 180 degrees. By varying one or more of L1, L2 and alpha, the expanded distance between the movable surfaces can be varied as needed or desired. For example, the expanded distance between the movable surfaces can be about 1.5 inches, about 1.55 inches, about 1.6 inches, about 1.65 inches, about 1.7 inches, about 1.75 inches, about 1.8 inches, about 1.85 inches, about 1.9 inches, about 1.95 inches, about 2.0 inches, etc.

Although FIGS. 7-9 illustrate the joints of the angles being oriented inwardly and thereby adjacent, the joints of the angles can be oriented outwardly so as to be set apart from each other. As should be evident to one of skill in the art, the hinges and joints are intended to be oriented and configured such that the surfaces 260, 265 can move in a following-movement of a patient's teeth during a mouth opening movement of the patient.

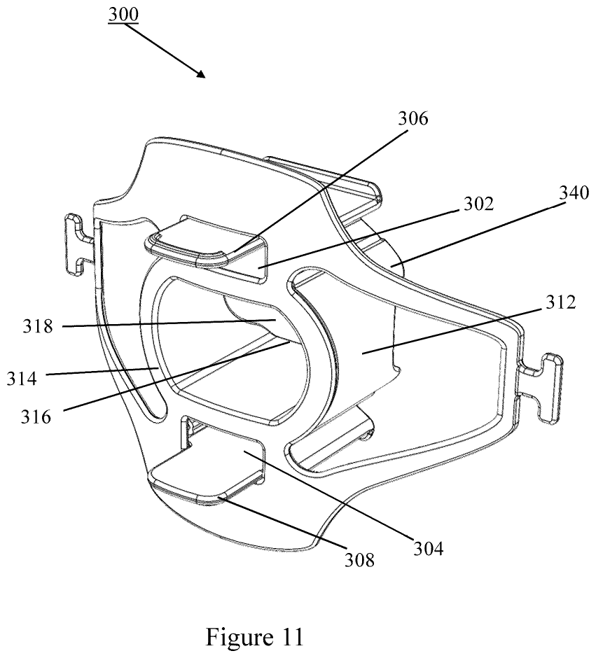

Referring to FIGS. 11 and 12, a third embodiment of a modified bite block 300 includes a tubular body or barrel 312 that defines a channel 318 that passes between proximal and distal openings 314, 316, respectively. The bite block 300 includes many of the features of the bite block 200, such as a front plate, upper and lower surfaces of the barrel 312. upper and lower walls, lateral wings, and clips. A flexible, expandable member 340 is positioned around the outer surfaces of the barrel 312 (i.e., around the side, upper and lower surfaces).

There are two primary difference between bite block 200 and bite block 300. The first primary difference is a pair of openings, an upper opening 302 and a lower opening 304 in the upper and lower portions, respectively, of the front plate. The second primary difference is a pair of prongs, an upper prong 306 and a lower prong 308, which each extend from the flexible, expandable member 340 through the openings 302, 304, respectively. These differences between the bite block 200 and bite block 300 permits a physician to grasp the prongs 306, 308, pull them in the direction of each other and thereby ease insertion of the bite block into a patient's mount. Similarly, once the bite block is in position within a patient's mouth, the physician can grasp the prongs, pull them in the direction of each other and thereby ease removal of the bite block from the patient's mouth.

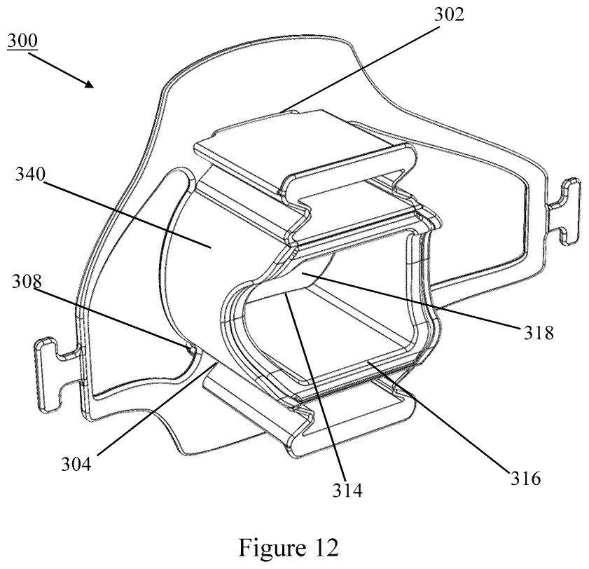

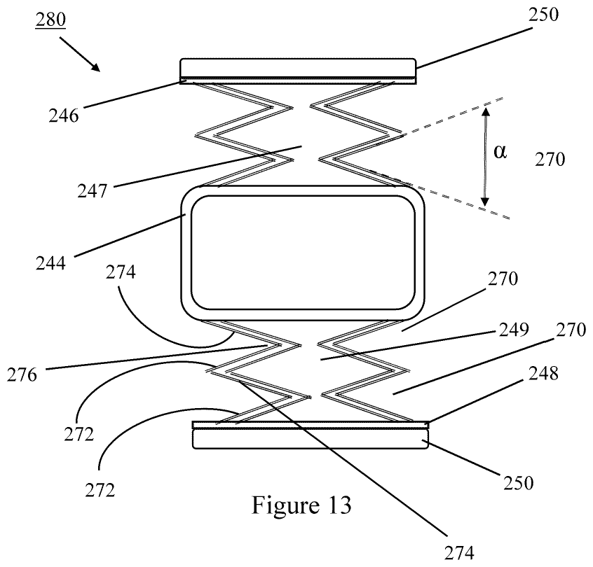

Referring to FIG. 13, in another variation of the configurations of FIGS. 3-12, where a pair of opposing hinges are positioned with each movable surface, each opposite side of each movable surface may include multiple hinges, i.e., two or more hinges, such that each hinge is made up of multiple pairs of walls 272, 274 with multiple angled joints. In the flexible member 280, which differs from the flexible member 240, each movable surface is biased against the tubular portion 244 by a pair of hinges that includes three angled joints. The length of each wall 272, 274 and angle formed between may be varied to modify the properties of the movable surfaces, such as rapidity of movement, distance of movement, resistance to movement, etc.

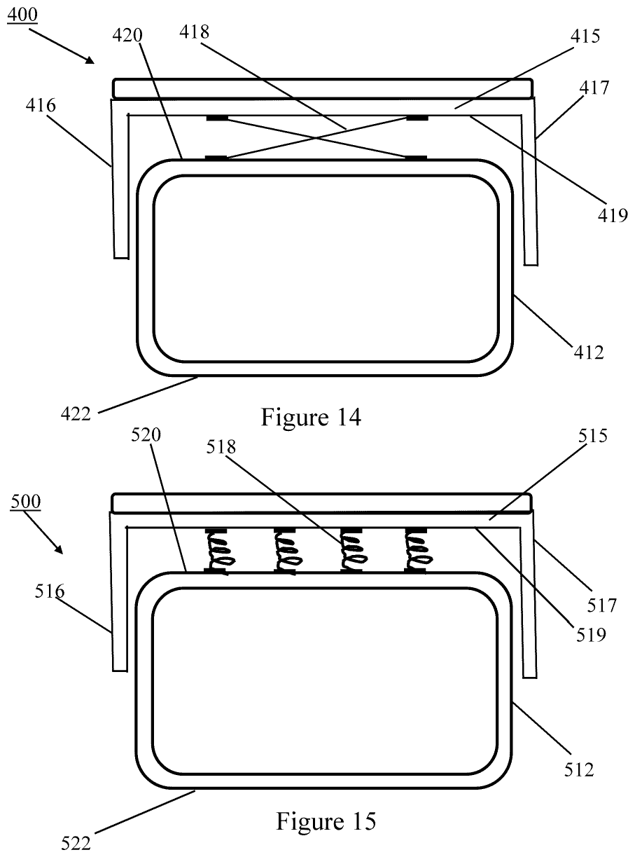

Referring to FIG. 14, in a fourth embodiment a modified bite block 400 includes the tubular body or barrel 412 with the modification to include an upper movable surface 415. Optionally, the upper movable surface 415 includes opposing surfaces 416, 417 that are generally perpendicular to the upper movable surface 415. The upper movable surface 415 is mounted to the top outer surface 420 using one or more elastic members 418. The elastic member 418 may be an elastic strip, hinge, spring or the like. The elastic member has the property of being capable of being resiliently deformed by the application of force but elastic so that the elastic member returns to its original configuration. For example, an elastic metal strip can be resiliently deformed by the application of a compressible force but upon removal or reduction in the force, the elastic strip returns or attempts to return to its original configuration. FIG. 14 illustrates a pair or elastic strips 418 having opposite ends being mounted to the upper surface 420 and a lower surface 419 of the upper movable surface 415, respectively.

When a patient's upper teeth rest upon the upper movable surface 415 and the patient's lower teeth rest upon the lower surface 422, the upper movable surface 415 will move with the movement of the patient's upper teeth. This will prevent the bite block from being dislodged from the patient's mouth during jaw opening. It should be understood that in another embodiment a second movable surface structure is positioned adjacent to the lower surface such that a patient's upper and lower teeth will be positioned on a movable surface.

FIG. 15 is an illustration of a fifth embodiment of a modified bite block 500 with a movable upper surface. The modified bite block 500 includes the tubular body 512 with the modification to include an upper movable surface 515. Optionally, the upper movable surface 515 includes opposing surfaces 516, 517 that are generally perpendicular to the upper movable surface 515. The upper movable surface 515 is mounted to the top outer surface 520 using one or more elastic members 518. FIG. 15 illustrates the elastic members 518 being in the form of an elastic spring. The elastic spring 518 has the property of being capable of being compressed by the application of force but elastic so that the spring returns to the original configuration upon removal of the applied force. For example, an elastic metal spring can be resiliently deformed by the application of a compressible force but upon removal or reduction in the force, the spring returns or attempts to return to its original configuration. FIG. 15 illustrates multiple elastic springs 518 having opposite ends being mounted to the upper surface 515 and a lower surface 519 of the upper movable surface 515, respectively.

When a patient's upper teeth rest upon the upper movable surface 515 and the patient's lower teeth rest upon the lower surface 522, the upper movable surface 515 will move with the movement of the patient's upper teeth. This will prevent the bite block from being dislodged from the patient's mouth during jaw opening. It should be understood that in another embodiment a second movable surface structure is positioned adjacent to the lower surface such that a patient's upper and lower teeth will be positioned on a movable surface.

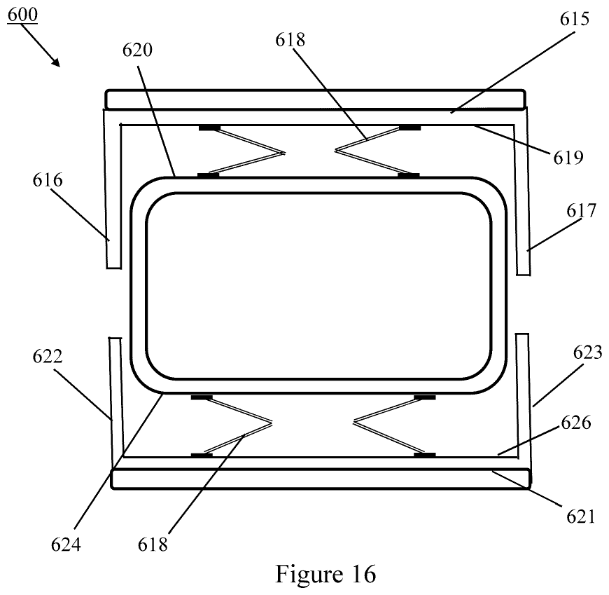

FIG. 16 is an illustration of a sixth embodiment of the modified endoscopic bite block. In contrast to the proceeding embodiments, the endoscopic bite block 600 of FIG. 16 includes an upper movable surface 615 and a lower movable surface 621. Optionally, one or both of the upper movable surface 615 and the lower movable surface 621 includes opposing surfaces 616, 617 and 622, 623, respectively, that are generally perpendicular to the movable surface 615, 621. The upper movable surface 615 is mounted to the top outer surface 620 using one or more elastic members 618 and the lower movable surface 621 is mounted to the lower outer surface 624 using one or more elastic members 618. FIG. 16 illustrates the elastic members 618 being in the form of elastic hinges 618. The elastic hinge 618 has the property of being capable of being compressed by the application of force but elastic so that the hinge returns to the original configuration upon removal of the applied force. For example, an elastic metal hinge can be resiliently deformed by the application of a compressible force but upon removal or reduction in the force, the hinge returns or attempts to return to its original configuration. FIG. 16 illustrates one set of multiple elastic hinges 618 having opposite ends being mounted to the upper surface 615 and a lower surface 619 of the upper movable surface 615, respectively. FIG. 16 also illustrates a second set of multiple elastic hinges 618 having opposite ends being mounted to the lower surface 624 and a lower movable surface 626 of the lower movable surface 621, respectively.

When a patient's upper teeth rest upon the upper movable surface 615 and the patient's lower teeth rest upon the lower movable surface 621, the movable surfaces will move with the movement of the patient's upper and lower teeth, respectively. This capability will prevent the bite block from being dislodged from the patient's mouth during jaw opening.

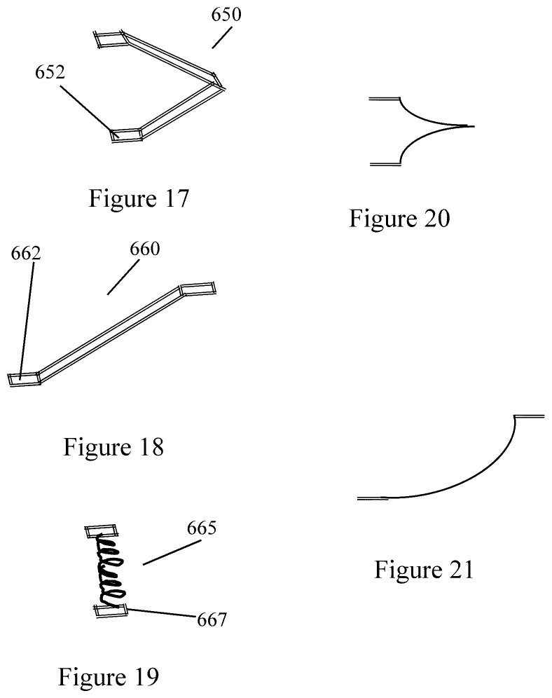

FIG. 17 illustrates an elastic hinge 650 that includes mounting ends 652 that are used to mount the hinge to the movable and stationary surfaces of the block bite. FIG. 18 illustrates an elastic strip 660 that includes mounting ends 662 that are used to mount the hinge to the movable and stationary surfaces of the block bite. FIG. 19 illustrates an elastic spring 665 that includes mounting ends 667 that are used to mount the hinge to the movable and stationary surfaces of the block bite. The hinge, strip or spring may be made of an elastic metal or polymer. For example, the elastic metal can be a superelastic metal such as nitinol, a nickel-titanium alloy. Other known elastic and superelastic metals and polymers may be used with the primary limitation being that the material is suitable for contact with the human body, in particular the mouth.

The hinge or strip may be of a variety of geometries. For example, FIGS. 17 and 18 illustrate the elastic member being generally straight. However, the hinge or strip may be bowed, as shown in FIGS. 20 and 21, respectively.

The elastic member may be of different degrees of elasticity and stiffness. The objective of the device is for the movable surface(s) to follow movement of the teeth resting upon that component. In other words, when during jaw opening the movable surface should stay in contact with the teeth to the extent possible to prevent malpositioning of the bite block. This objective can be achieved by the selection of the material or the physical characteristics of the elastic member. For example, the elastic member can be made of a thicker or thinner member to control rigidity, elasticity and the speed at which the elastic member expands. These properties must be balanced against the force exerted against the patient's teeth and jaw. The force exerted should not be so great as to be uncomfortable to the patient.

The movable surfaces, separately or combined, may be configured to travel approximately one to two inches during jaw opening. Reports in the literature indicate that individuals can open their mouth approximately 1.4 to 2.2 inches (3.5 to 5.5 cm). As described above, the tubular body or barrel 12 of a conventional endoscopic bite block includes top and bottom outer surfaces 30, 32. For a bite block configured to receive a 54F endoscope, the distance between the outer surfaces is approximately one inch, thereby requiring the patient to open the jaws at least one inch to position the conventional bite block between the upper and lower teeth. In one embodiment, the movable surface(s) used to modify the conventional bite block will add approximately 1/16 inch to ⅛ inch in thickness to the inventive bite block, which will only slightly increase the amount the patient must open their jaws. If the patient may open their jaws up to 2.2 inches, the elastic members are configured to cause the movable surfaces to travel up to approximately 1 to 1.5 inches.

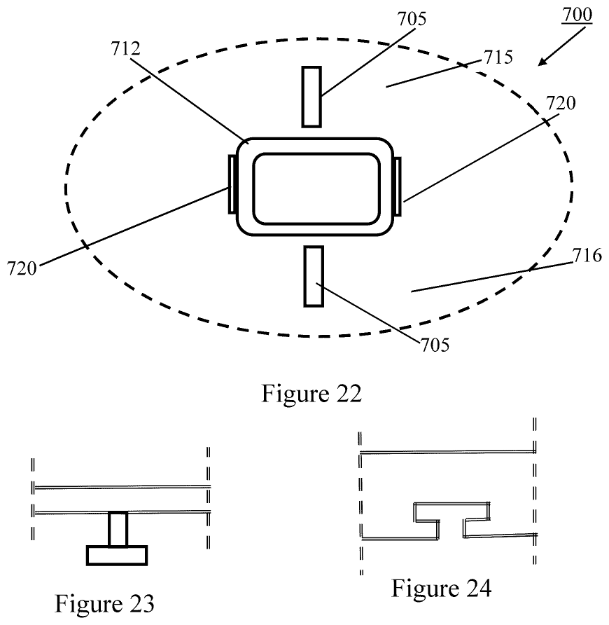

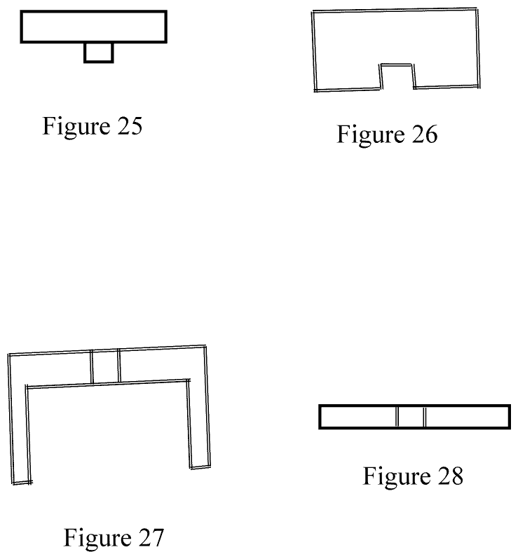

The modified endoscopic bite block can be implemented with mating groves and flanges or tabs to hold the movable surface member in place or guide the movement of the movable surface member. FIG. 22 schematically illustrates a modified bite block 700 with placement of one or more grooves, flanges or tabs. The bite block can include one or more grooves, flanges or tabs 705 placed on one of both of the upper and lower walls 715, 716. For example, the groove, flange or tab may be placed on the upper wall 715 or the lower wall 716 or both walls. Alternatively, or in addition, a groove, flange or tab 720 may be placed on one or both of the outside surfaces of the side of the tubular member or barrel 712. Referring also to FIGS. 22-27, the groove, flange or tab 705, 720 may be in the form of a T-shaped flange (FIG. 23) or a groove (FIG. 24). The flange of FIG. 23 can interact with the channel illustrated in FIG. 24 to retain the movable surface to the bite block. The groove of FIG. 26 can interact with the tab illustrated in FIG. 25 to guide the movement of the movable surface along the bite block.

Referring to FIGS. 27 and 28, the movable surface can be implemented with the groove, flange or tab and include optional side walls (FIG. 27) or be free of the side walls (FIG. 28). The grooves, flanges and tabs can be on both side surfaces and/or an edge surface of the movable surface so that the movable surface is guided or retained to the bite block.

The hinge, spring, or strip can be retained to the movable surface member and the tubular or barrel member by use of conventional slots that an end can be inserted into and kept in place using techniques well known in the art, e.g., a friction fit or application of an adhesive.

The flexible member and the movable surface member as well as the bite block itself may be made using conventional plastic molding techniques, including injection molding and 3-D printing.

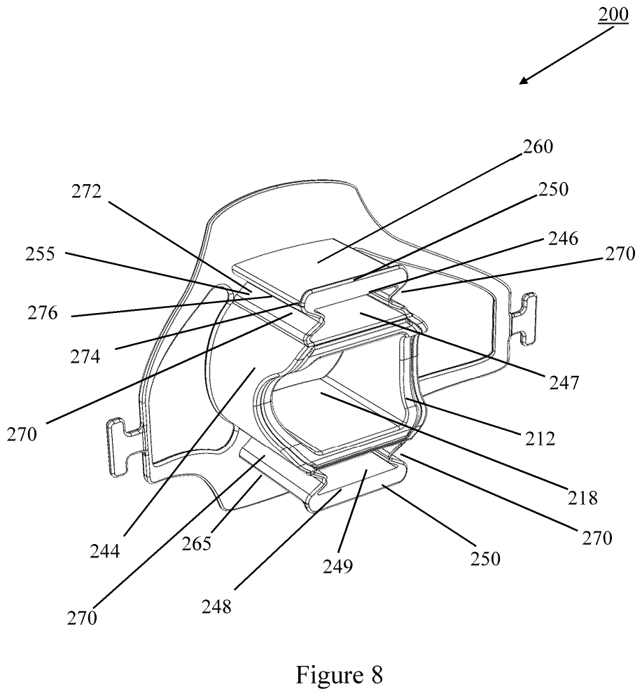

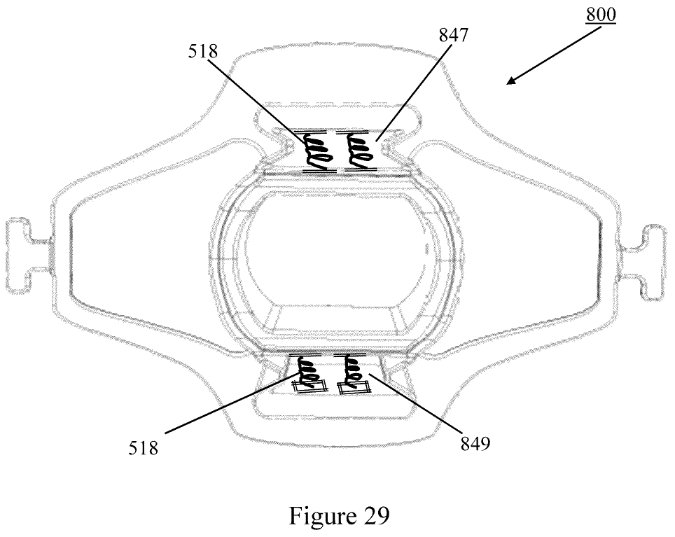

Although a number of variations of a modified endoscopic bite block have been disclosed herein, it should be understood that various aspects of each variation can be combined. For example, the endoscopic bite block of FIGS. 3-13 can include one or more of the elastic member 418, elastic spring 518, and elastic hinge 618 disclosed in FIGS. 14, 15 and 16, respectively. For example, referring to FIG. 29, an additional embodiment of a bite block 800 is based on combining the second embodiment of an endoscopic bite block illustrated in FIG. 8 with the addition of one or more elastic springs 518 positioned within the channels 847, 849. Similar combinations of the various elastic members and various flexible member can be combined by one of skill in the art. The flexible member can be injection molded around the elastic member to vary the elasticity and ability for the surfaces to follow the movement of the patient's teeth. The flexible member can be a separate item that is injection molded around the tubular member or separately molded and physically placed around the tubular member.

What is claimed is:

1. A bite block for using during endoscopic procedures, the bite block comprising:
a tubular body having an internal lumen configured to receive an endoscope, and having an upper outer surface and a lower outer surface;
a front plate comprising an upper wall portion and a lower wall portion, both connected to the tubular body and the upper wall extending in a first direction and the lower wall extending in a second generally opposite direction;
at least one movable surface member mounted to at least one of the upper outer surface and the lower outer surface of the tubular body and configured to move relative to the tubular body; and
at least one elastic member joining the movable surface member to the tubular body, whereby the elastic member is configured to exert a force against movement of the movable surface member in the direction of the tubular body.

2. The bite block of claim 1, further comprising a strap or band connected to the bite block and configured to retain the bite block to a subject's head.

3. The bite block of claim 1, further comprising at least one of an upper ridge and a lower ridge extending from the upper movable surface member and the lower movable surface member, respectively, and configured to interact with the subject's teeth to resist the bite block from sliding out of a subject's mouth in use.

4. The bite block of claim 1, wherein the bite block comprises an upper movable surface member and a lower movable surface member.

5. The bite block of claim 1, wherein the movable surface member comprises a first portion having a first end and a second opposite end and is generally flat.

6. The bite block of claim 5, wherein the movable surface member further comprises a first side portion extending from the first end and generally perpendicular to the first portion and a second side portion extending from the second end and generally perpendicular to the first portion and parallel to the first side portion.

7. The bite block of claim 1, wherein the flexible member comprises one or more of an elastic spring, an elastic hinge and an elastic strip.

8. The bite block of claim 1, wherein the flexible member comprises one or more elastic hinges.

9. The bite block of claim 1, wherein the front plate comprises one or more of a groove, flange or tab and the movable surface comprises one or more of a groove, flange or tab configured to mate with the groove, flange or tab on the front plate.

10. The bite block of claim 9, wherein the mating of the groove, flange or tab between the front plate and movable surface guides the movement of the movable surface relative to the front plate or retains the movable surface to the front plate during movement of the movable surface relative to the front plate.

11. The bite block of claim 1, wherein an outer surface of the tubular body comprises one or more of a groove, flange or tab and the movable surface comprises one or more of a groove, flange or tab configured to mate with the groove, flange or tab on the tubular body.

12. The bite block of claim 11, wherein the mating of the groove, flange or tab between the tubular body and movable surface guides the movement of the movable surface relative to the tubular body or retains the movable surface to the tubular body during movement of the movable surface relative to the tubular body.

13. A method of performing an endoscopic procedure on a patient, the method comprising:
providing a bite block, wherein the bite block comprises:
a tubular body having an internal lumen configured to receive an endoscope, and having an upper outer surface and a lower outer surface,
a front plate comprising an upper wall portion and a lower wall portion, both connected to the tubular body and the upper wall extending in a first direction and the lower wall extending in a second generally opposite direction,
at least one movable surface member mounted to at least one of the upper outer surface and the lower outer surface of the tubular body and configured to move relative to the tubular body, and
at least one elastic member joining the movable surface member to the tubular body, whereby the elastic member is configured to exert a force against movement of the movable surface member in the direction of the tubular body;
positioning the bite block within the patient's mouth such that the patient's upper teeth are in contact with the upper outer surface and the patient's lower teeth are in contact with the lower outer surface; and
introducing an endoscope into the internal lumen of the tubular body,
wherein when the upper surface follows the patient's upper teeth and the lower surface follows the patient's lower teeth when the patient yawns during the endoscopic procedure.

14. The method of claim 13, wherein the bite block further comprises a strap or band connected to the bite block and the strap or band is positioned around the back of the patient's head.

15. A method of making an endoscopic bite block, the method comprising:
providing a bite block, wherein the bite block comprises a tubular body having an internal lumen configured to receive an endoscope, and having an upper outer surface and a lower outer surface, and a front plate comprising an upper wall portion and a lower wall portion, both connected to the tubular body and the upper wall extending in a first direction and the lower wall extending in a second generally opposite direction; and
injection molding a flexible member to the bite block, the flexible member comprising at least one movable surface member mounted to at least one of the upper outer surface and the lower outer surface of the tubular body and configured to move relative to the tubular body, and at least one elastic member joining the movable surface member to the tubular body, whereby the elastic member is configured to exert a force against movement of the movable surface member in the direction of the tubular body.
